(19) United States
(12) Patent Application Publication
(10) Pub. No.: US 2023/0023456 A1
(43) Pub. Date: Jan. 26, 2023

(54) RECOVERY OF DEEPLY DISCHARGED IMPLANTABLE BATTERY

(71) Applicant: Cochlear Limited, Macquarie University, NSW (AU)

(72) Inventors: Thomas Cooney, Redfern, NSW (AU); Padraig Hurley, Frenches Forest, NSW (AU); Tim Blanchaert, Geel (AU)

(21) Appl. No.: 17/757,733

(22) PCT Filed: Dec. 7, 2020
(86) PCT No.: PCT/IB2020/061598

§ 371 Date: Jun. 20, 2022

Related U.S. Application Data
(60) Provisional application No. 62/958,138, filed on Jan. 7, 2020.

(51) Int. Cl. A61N 1/378; A61N 1/36; H02J 50/10; H02J 7/00

(52) U.S. Cl. CPC A61N 1/3787; A61N 1/36038; H02J 50/10; H02J 7/00712; H02J 2310/23

(57) ABSTRACT

An apparatus includes at least one housing configured to be implanted on or within a recipients body. The apparatus further includes first circuitry configured to wirelessly receive power from a device external to the recipients body, second circuitry configured to provide stimulation signals to a portion of the recipients body, and at least one power storage device having a discharged state in which the at least one power storage device is discharged to a voltage below a minimum operating voltage of the at least one power storage device. The apparatus further includes third circuitry configured to, while the at least one power storage device is in the discharged state, controllably distribute the power simultaneously to both the second circuitry and the at least one power storage device.

### BACKGROUND

### Field

The present application relates generally to systems and methods for facilitating wireless power transmission and distribution, and more specifically, for facilitating wireless power transmission between an external portion and an implanted portion of an implanted medical system and distribution of the power by the implanted portion.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises at least one housing configured to be implanted on or within a recipient's body. The apparatus further comprises first circuitry within the at least one housing, the first circuitry configured to wirelessly receive power from a device external to the recipient's body. The apparatus further comprises second circuitry within the at least one housing, the second circuitry configured to provide stimulation signals to a portion of the recipient's body. The apparatus further comprises at least one power storage device within the at least one housing. The at least one power storage device has a discharged state in which the at least one power storage device is discharged to a voltage below a minimum operating voltage of the at least one power storage device. The apparatus further comprises third circuitry within the at least one housing. The third circuitry is configured to, while the at least one power storage device is in the discharged state, controllably distribute the power simultaneously to both the second circuitry and the at least one power storage device.

In another aspect disclosed herein, a method comprises receiving electric power via a magnetic induction link. The electric power is received by circuitry implanted on or within a recipient's body from circuitry external to the recipient's body. The method further comprises, while at least one power storage device implanted on or within the recipient's body is discharged to a voltage below a minimum operating voltage of the at least one power storage device, storing a first portion of the received electric power in the at least one power storage device. The method further comprises simultaneously with said storing the first portion, using a second portion of the received electric power to operate at least one actuator implanted on or within the recipient's body.

In another aspect disclosed herein, an apparatus comprises a power transfer circuit configured to wirelessly receive power. The apparatus further comprises at least one battery configured to store at least a first portion of the power received by the power transfer circuit. The at least one battery has a discharged state and at least one non-discharged state. The apparatus further comprises at least one actuator configured to operate using power from the power transfer circuit and/or power from the at least one battery. The apparatus further comprises a controller configured to, while the at least one battery is in the discharged state, controllably distribute the power received by the magnetic induction circuitry simultaneously to both the at least one battery and the at least one actuator.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 schematically illustrates an example apparatus in accordance with certain implementations described herein;

FIGS. 3A-3B schematically illustrate example features of the third circuitry of two example apparatus in accordance with certain implementations described herein; and

FIG. 4 is a flow diagram of an example method in accordance with certain implementations described herein.

### DETAILED DESCRIPTION

In certain systems using a lithium-ion battery, the battery can enter a discharged state in which the battery voltage is below a minimum operating voltage of the battery. Because the battery in this discharged state cannot provide electrical power to the system, the system is unable to operate (e.g., during a pre-charge and/or re-charge operation) until the battery stores sufficient electrical power to be in a non-discharged state. In certain implementations disclosed herein, the system includes circuitry that is configured to, when the battery is in the discharged state (e.g., during a pre-charge and/or re-charge operation), controllably parse the incoming electrical power to both the battery and the system so that the system is able to operate while the battery is in the discharged state. The circuitry can ensure that the supply voltage that powers the battery management circuitry and other functions of the system during pre-charge remains above a minimum operating value to support system operation unrelated to battery measurement (e.g., stimulation by an implantable actuator). Such functionality would not be found in systems that are not expected to be used during the pre-charge operation (e.g., a battery power tool; electronics systems that are in reset during a pre-charge operation).

The teachings detailed herein are applicable, in at least some implementations, to any type of implantable medical device (e g, implantable sensory prostheses) comprising a first portion (e.g., external to a recipient) and a second portion (e.g., implanted on or within the recipient), the first portion configured to wirelessly transmit power to the second portion. For example, the implantable medical device can comprise an auditory prosthesis system utilizing an external sound processor configured to transcutaneously provide power to an implanted assembly (e.g., comprising an actuator). In certain such examples, the external sound processor is further configured to transcutaneously provide data (e.g., control signals) to the implanted assembly that responds to the data by generating stimulation signals that are perceived by the recipient as sounds. Examples of auditory prosthesis systems compatible with certain implementations described herein include but are not limited to: electro-acoustic electrical/acoustic systems, cochlear implant devices, implantable hearing aid devices, middle ear implant devices, Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components Implementations can include any type of medical device that can utilize the teachings detailed herein and/or variations thereof.

Merely for ease of description, apparatus and methods disclosed herein are primarily described with reference to an illustrative medical device, namely a cochlear implant. However, the teachings detailed herein and/or variations thereof may also be used with a variety of other medical devices that provide a wide range of therapeutic benefits to recipients, patients, or other users. In some implementations, the teachings detailed herein and/or variations thereof can be utilized in other types of implantable medical devices beyond auditory prostheses. For example, apparatus and methods disclosed herein and/or variations thereof may also be used with one or more of the following: vestibular devices (e.g., vestibular implants); visual devices (e.g., bionic eyes); visual prostheses (e.g., retinal implants); sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; etc. The concepts described herein and/or variations thereof can be applied to any of a variety of implantable medical devices comprising an implanted component configured to use magnetic induction to receive power (e.g., transcutaneously) from an external component and to store at least a portion of the power in at least one power storage device (e.g., battery). The implanted component can also be configured to receive control signals from the external component (e.g., transcutaneously) and/or to transmit sensor signals to the external component (e.g., transcutaneously) while receiving power from the external component. In still other implementations, the teachings detailed herein and/or variations thereof can be utilized in other types of systems beyond medical devices utilizing magnetic induction for wireless power transfer. For example, such other systems can include one or more of the following: consumer products (e.g., smartphones; “internet-of-things” or IoT devices) and electric vehicles (e.g., automobiles).

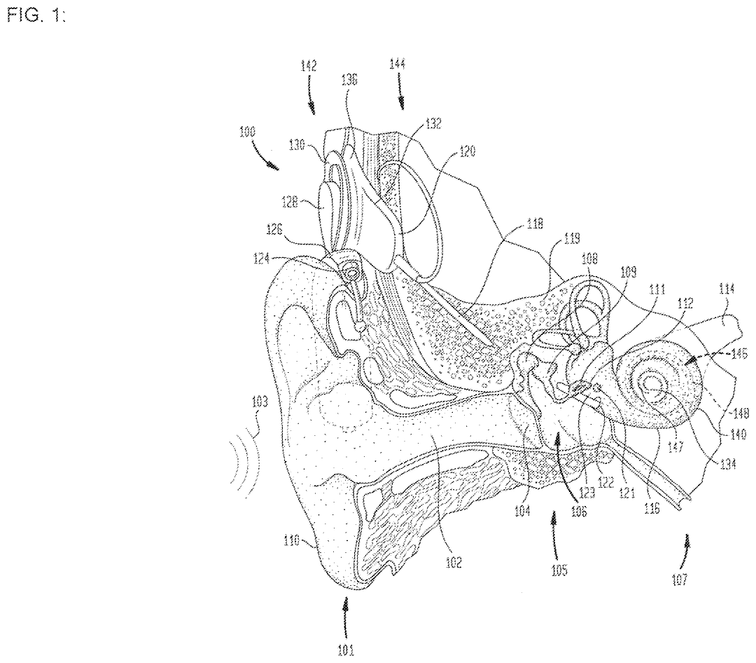

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain implementations described herein can replace the external microphone assembly 124 shown in FIG. 1 with a subcutaneously implantable assembly comprising an acoustic transducer (e.g., microphone), as described more fully herein.

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1)(e.g., audio ports, such as a Direct Audio Input (DAI); data ports, such as a Universal Serial Bus (USB) port; cable ports, etc.). In the example of FIG. 1, the sound processing unit 126 is a behind-the-ear (BTE) sound processing unit configured to be attached to, and worn adjacent to, the recipient's ear. However, in certain other implementations, the sound processing unit 126 has other arrangements, such as by an OTE processing unit (e.g., a component having a generally cylindrical shape and which is configured to be magnetically coupled to the recipient's head), a mini or micro-BTE unit, an in-the-canal unit that is configured to be located in the recipient's ear canal, a body-worn sound processing unit, etc.

The sound processing unit 126 of certain implementations includes a power source (not shown in FIG. 1)(e.g., battery), a processing module (not shown in FIG. 1)(e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementation of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted implementation of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are hermetically sealed within a biocompatible housing, sometimes collectively referred to as a stimulator/receiver unit. The internal receiver unit 132 comprises at least one internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates stimulation signals (e.g., electrical stimulation signals; optical stimulation signals) based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 can be implanted at least in the basal region 116, and sometimes further. For example, the stimulation assembly 118 can extend towards an apical end of the cochlea 140, referred to as the cochlea apex 134. In certain circumstances, the stimulation assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). The stimulation elements 148 are longitudinally spaced from one another along a length of the elongate body of the stimulation assembly 118. For example, the stimulation assembly 118 can comprise an array 146 comprising twenty-two (22) stimulation elements 148 that are configured to deliver stimulation to the cochlea 140. Although the array 146 of stimulation elements 148 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

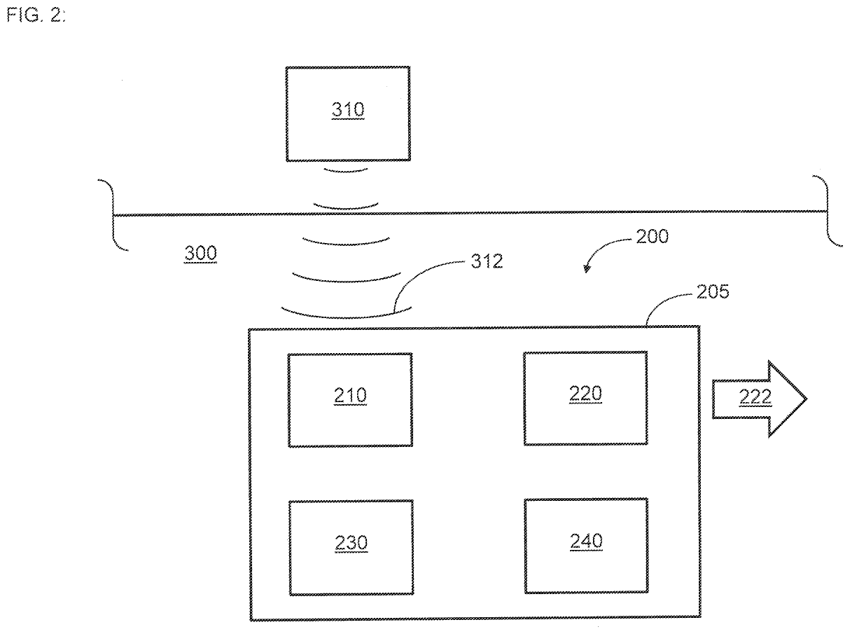

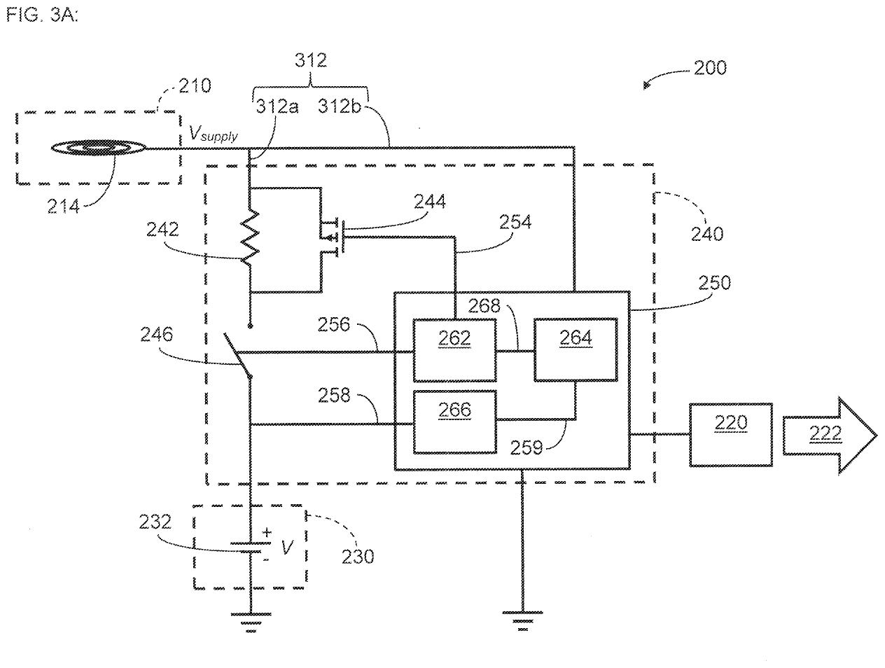

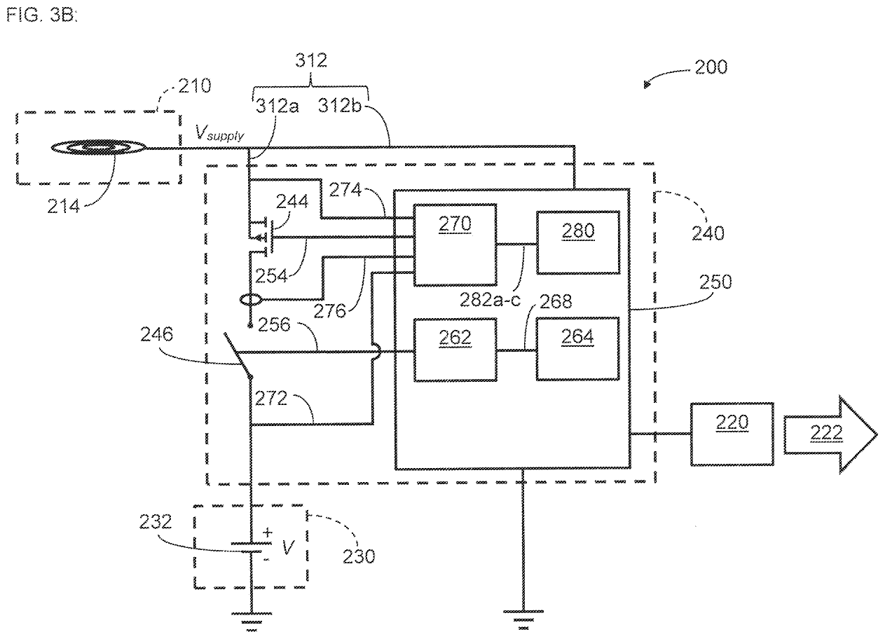

FIG. 2 schematically illustrates an example apparatus 200 in accordance with certain implementations described herein. FIGS. 3A-3B schematically illustrate additional features of two example apparatus 200 in accordance with certain embodiments described herein. The apparatus 200 comprises at least one housing 205 configured to be implanted on or within a recipient's body 300. The apparatus 200 further comprises first circuitry 210 within the at least one housing 205, the first circuitry 210 configured to wirelessly receive power 312 from a device 310 external to the recipient's body 300. The apparatus 200 further comprises second circuitry 220 within the at least one housing 205, the second circuitry 220 configured to provide stimulation signals 222 to a portion of the recipient's body 300. The apparatus 200 further comprises at least one power storage device 230 within the at least one housing 205, the at least one power storage device 230 having a discharged state in which the at least one power storage device 230 is discharged to a voltage below a minimum operating voltage of the at least one power storage device 230. The apparatus 200 further comprises third circuitry 240 within the at least one housing 205, the third circuitry 240 configured to, while the at least one power storage device 230 is in the discharged state, controllably distribute the power 312 simultaneously to both the second circuitry 220 and the at least one power storage device 230.

In certain implementations, the apparatus 200 is an implanted portion of a medical system (e.g., a portion of the medical system that is implanted on or within the recipient) and the device 310 from which the apparatus 200 wirelessly receives the power 312 comprises an external portion of the medical system (e.g., a portion worn by the recipient; a portion that is configured to be repeatedly attached and detached from the apparatus 200 and/or the recipient). For example, the device 310 can comprise an external portion (e.g., a sound processing unit 126) of an auditory prosthesis 100 (e.g., a cochlear implant system) and the apparatus 200 can comprise an implanted stimulator unit 120.

In certain implementations, the housing 205 comprises at least one biocompatible material (e.g., polymer; silicone; titanium; titanium alloy) and is configured to be at least partially implanted on or within the recipient (e.g., a region within the housing 205 is hermetically sealed from a region outside the housing 205). Besides containing the first circuitry 210, the second circuitry 220, the at least one power storage device 230, and the third circuitry 240, the housing 205 of certain implementations is configured to further contain at least one of: communication circuitry (e.g., magnetic inductive RF data transfer circuitry; at least one antenna configured to be operationally coupled to a corresponding at least one antenna of the external device 310) configured to communicate data signals to and/or from the external device 310; processing circuitry configured to process data signals from the external device 310; a magnetic material configured to interact with a magnet of the external device 310 to create an attractive magnetic force that adheres the external device 310 to the recipient's body 300 (e.g., holds the external device 310 in an operative position relative to the apparatus 200). For example, for an auditory prosthesis 100, the communication circuitry can be configured to receive data signals generated by a microphone 124 and transmitted to the apparatus 200 by a sound processing unit 126, and the processing circuitry can be configured to process the received data signals (e.g., utilizing digital processing techniques for frequency shaping, amplification, compression, and/or other signal conditioning, including conditioning based on recipient-specific fitting parameters). The second circuitry 220 can be configured to respond to the processed data signals by generating the stimulation signals 222 that are provided to a portion of the recipient's body 300 (e.g., to create a hearing percept).

In certain implementations, the first circuitry 210 comprises at least one electrically conductive power transfer coil 214 configured to be operationally coupled by magnetic induction to at least one corresponding electrically conductive power transfer coil of the external device. For example, the at least one power transfer coil 214 can comprise an electrically conductive conduit (e.g., wire; conductive trace on a printed circuit board). The at least one power transfer coil 214 is configured to generate electric power 312 in response to a time-varying magnetic field generated by the at least one power transfer coil of the external device 300. For example, the time-varying magnetic field and the electric power can have a frequency in a range of 100 kHz to 100 MHz (e.g., 5 MHz; 6.78 MHz; 12 MHz; 49 MHz). In certain implementations in which the apparatus 200 comprises an implanted portion of a medical system, the power transfer is in a range of 1 mW to 500 mW. In certain other implementations, the power transfer is in a range of 1 W to 1 kW (e.g., for an apparatus 200 comprising a consumer device and/or an “internet-of-things” or IoT device) or in a range of 1 kW to 100 kW (e.g., for an apparatus comprising a vehicle).

In certain implementations in which the apparatus 200 is an implanted portion of a medical system, the second circuitry 220 is configured to provide stimulation signals 222 to a portion of the recipient's body 300. For example, for a cochlear implant auditory prosthesis 100 (see, e.g., FIG. 1), the second circuitry 220 can comprise an implanted stimulator unit 120 and a stimulation assembly 118 (e.g., comprising an elongate array 146 of stimulation elements 148) configured to generate the stimulation signals 222 (e.g., electrical stimulation signals; optical stimulation signals) in response to received data signals and to deliver the stimulation signals 222 to the recipient (e.g., to the cochlea 140 to create a hearing percept) via the elongate stimulation assembly 118. For another example, for an implanted visual prosthesis (e.g., retinal prosthesis), the second circuit 220 can comprise an array of stimulation elements configured to generate the stimulation signals 222 in response to received data signals and to deliver the stimulation signals 222 to the recipient (e.g., to the optic nerve or other portion of the recipient's vision system to create a vision percept). In still another example, for an implanted cardiac pacemaker, the second circuit 220 can comprise one or more stimulation elements (e.g., electrodes) configured to generate the stimulation signals 222 in response to received data signals and to deliver the stimulation signals 222 to the recipient (e.g., to selected portions of the heart to modify and/or control the heart's operation).

In certain implementations, the at least one power storage device 230 comprises at least one battery 232 having a discharged state in which the at least one battery 232 is discharged to a voltage below a minimum operating voltage of the at least one battery 232. While in the discharged state, the at least one battery 232 cannot provide electric current to a load without causing damage to the at least one battery 232 (e.g., reducing a usable life of the at least one battery 232). In order to transition the at least one battery 232 from the discharged state to a non-discharged state, the at least one battery 232 can be pre-charged with an electric current at or below a predetermined limit to bring its voltage above the minimum operating voltage such that the at least one battery 232 can provide electric current to the load.

In certain implementations, the at least one battery 232 can comprise at least one lithium-ion battery having a minimum operating voltage (e.g., in a range of 2.5 V to 3 V; 2.7 V). The voltage of the at least one battery 232 in the discharged state can be 0 V (e.g., due to self-discharge over a sufficiently long period of inactivity). For example, for a lithium-ion battery used in a totally implantable cochlear implant (“TICI”), the lithium-ion battery can enter the discharged state if the recipient stops using the lithium-ion battery for a sufficiently long period of time such that the self-discharge brings the voltage of the lithium-ion battery below the minimum operating voltage.

In certain such implementations, the third circuitry 240 is configured to ensure that the low voltage of the at least one battery 232 while in the discharged state (e.g., during pre-charge and/or re-charge) does not prevent the TICI being used in external hearing mode to provide a hearing percept to the recipient. For example, the third circuitry 240 can be configured to controllably parse the received electrical power 312 between the at least one battery 232 and the second circuitry 220 so that the second circuitry 220 can operate while the at least one battery 232 is in the discharged state (e.g., providing a portion of the received electrical power 312 to the second circuitry 220 while maintaining an electrical charge current to the at least one battery 232 that is at or below a pre-charge current limit of the at least one battery 232).

FIGS. 3A-3B schematically illustrate example third circuitry 240 of two example apparatus 200 in accordance with certain implementations described herein. The third circuitry 240 is configured to, while the at least one power storage device 230 is in the discharged state, controllably distribute the electric power 312 (e.g., electrical current) simultaneously to both the second circuitry 220 and the at least one power storage device 230. The example third circuitry 240 of certain implementations comprises dedicated circuitry that controllably limits a charging current into the at least one power storage device 230, and allows a supply voltage V_{supply} of the first circuitry 210 (e.g., in a range of 2.6 V to 4 V) to remain above a reset voltage Vᵣₑₛₑₜ of the third circuitry 240 (e.g., in a range of 1 V to 3.5 V), which can be higher than the voltage V of the at least one power storage device 230 in the discharged state (e.g., in a range of 2.5 V to 3 V). For example, the charging current can be controllably limited by the example third circuitry 240 to be in a range of 5% to 10% of the capacity of the at least one power storage device 230 (e.g., the capacity in a range of 20 mA-hours to 50 mA-hours).

As schematically illustrated in FIG. 3A, the example third circuitry 240 comprises at least one resistor 242, at least one transistor 244, at least one switch 246, and control circuitry 250. The at least one resistor 242 and the at least one transistor 244 are in parallel electrical communication with one another, and the at least one switch is in series electrical communication with the at least one resistor 242 and the at least one transistor 244 and with the at least one power storage device 230 (e.g., the at least one battery 232). The at least one resistor 242 and the at least one transistor 244 are configured to receive at least a first portion 312a of the electrical power 312 (e.g., electrical current) from the first circuitry 210. The at least one transistor 244 (e.g., a MOSFET) is configured to selectively switch between an open state and a closed state (e.g., the at least one transistor 244 in the closed state forming an electrical short across the at least one resistor 242). The at least one switch 246 (e.g., a battery enable switch) is configured to selectively switch between an open state and a closed state (e.g., the at least one switch 246 in the closed state providing the first portion 312a of the electrical power 312 to the at least one power storage device 230).

The control circuitry 250 is configured to receive at least a second portion 312b of the electrical power 312 from the first circuitry 210 and to controllably provide at least some of the second portion 312b of the electrical power 312 to the second circuitry 220. The control circuitry 250 is in electrical communication with the at least one transistor 244 and is configured to provide transistor control signals 254 (e.g., binary on/off signals) to the at least one transistor 244 and is in electrical communication with the at least one switch 246 and is configured to provide switch control signals 256 (e.g., binary on/off signals) to the at least one switch 246.

The control circuitry 250 of FIG. 3A comprises an input/output circuit 262, a microcontroller circuit 264, and an analog-to-digital converter circuit 266. The input/output circuit 262 is in electrical communication with the microcontroller circuit 264 and is configured to controllably transmit the transistor control signals 254 to the at least one transistor 244 and the switch control signals 256 to the at least one switch 246 in response to control signals 268 received from the microcontroller circuit 264. The analog-to-digital converter circuit 266 is in electrical communication with the at least one power storage device 230 and the microcontroller circuit 264. The analog-to-digital converter circuit 266 is configured to receive analog sensor signals 258 (e.g., battery voltage sense signals) indicative of the voltage V (e.g., sensed with respect to ground or Vₛₛ) of the at least one power storage device 230 (e.g., the voltage of the at least one battery 232) and to generate and provide digital sensor signals 259 to the microcontroller circuit 264. The microcontroller circuit 264 is configured to generate the control signals 268 in response, at least in part, to the voltage V of the at least one power storage device 230 (e.g., in response to the digital sensor signals 259) such that the third circuitry 240 is configured to generate the transistor control signals 254 in response, at least in part, to the voltage V of the at least one power storage device 230. In certain implementations, the microcontroller circuit 264 is further configured to controllably provide at least some of the second portion 312b of the electrical power 312 to the second circuitry 220.

In certain implementations, the at least one resistor 242 is between the first circuitry 210 and the at least one power storage device 230 and is controllably connected and disconnected to the at least one power storage device 230 (e.g., by the at least one switch 246) such that the first portion 312a of the electrical power 312 (e.g., a pre-charge current) into the at least one power storage device 230 is controllably limited. Simultaneously with the first portion 312a of the electrical power 312 being provided to the at least one power storage device 230, at least some of the second portion 312b of the electrical power 312 is provided to the second circuitry 220 (e.g., a main supply voltage V_{supply} of the first circuitry 210 is significantly higher than the voltage of the at least one battery 232, provided that sufficient electrical power 312 is available from the first circuitry 210).

In certain implementations, if the at least one power storage device 230 is to be used to power the second circuitry 220, the microcontroller circuit 264 turns on (e.g., closes) the at least one switch 246 (e.g., by transmitting control signals 268 to the input/output circuit 262 which transmits the corresponding switch control signals 256 to the at least one switch 246). The analog-to-digital converter circuit 266 senses the voltage V of the at least one power storage device 230 (e.g., the battery voltage) and transmits digital sensor signals 259 indicative of the voltage V to the microcontroller circuit 264. The microcontroller circuit 264 determines whether the voltage V is above a minimum operating voltage Vₘᵢₙ of the at least one power storage device 230 and whether the voltage V is above a reset voltage Vᵣₑₛₑₜ of the control circuitry 250. For example, the control circuitry 250 can comprise pre-programmed values of the minimum operating voltage Vₘᵢₙ and the reset voltage Vᵣₑₛₑₜ (e.g., in the microcontroller program code; in a programmable data storage device in operative communication with the microcontroller circuit 264).

If the voltage V of the at least one power storage device 230 is above both the minimum operating voltage Vₘᵢₙ and the reset voltage Vᵣₑₛₑₜ, the microcontroller circuit 264 turns on (e.g., activates) the at least one transistor 244 such that the at least one transistor 244 shorts the at least one resistor 242 (e.g., by transmitting control signals 268 to the input/output circuit 262 which transmits the corresponding transistor control signals 254 to the at least one transistor 244). In this way, the at least one transistor 244 enables charging of the at least one power storage device 230 (e.g., by flowing the first portion 312a of the electrical power 312 to the at least one power storage device 230 through the at least one transistor 244) and/or discharging of the at least one power storage device 230 (e.g., by flowing electrical power from the at least one power storage device 230 to the control circuitry 250 through the at least one transistor 244).

If the voltage V of the at least one power storage device 230 is below either the minimum operating voltage Vₘᵢₙ or the reset voltage Vᵣₑₛₑₜ, the microcontroller circuit 264 turns off (e.g., deactivates) the at least one transistor 244 (e.g., by transmitting control signals 268 to the input/output circuit 262 which transmits the corresponding transistor control signals 254 to the at least one transistor 244). When electrical power 312 is available from the first circuitry 210 under such conditions, the first portion 312a of the electrical power 312 flows through the at least one resistor 242 and the second portion 312b of the electrical power 312 flows to the control circuitry 250 for powering the second circuitry 220. In certain implementations, the resistance of the at least one resistor 242 is configured such that, for the smallest amount of electrical power expected to be available from the first circuitry 210 across expected operating conditions, the first portion 312a of the electrical power 312 flowing to the at least one power storage device 230 is limited such that the supply voltage V_{supply} is greater than the reset voltage Vᵣₑₛₑₜ of the control circuitry 250.

As schematically illustrated in FIG. 3B, the example third circuitry 240 comprises at least one transistor 244, at least one switch 246, and control circuitry 250. The at least one transistor 244 is configured to receive at least a first portion 312a of the electrical power 312 (e.g., electrical current) from the first circuitry 210. The at least one transistor 244 (e.g., a MOSFET) is configured to selectively provide the first portion 312a of the electrical power 312 to the at least one switch 246. The at least one switch 246 (e.g., a battery enable switch) is in series electrical communication with the at least one resistor 242 and the at least one transistor 244 and is configured to selectively provide the first portion 312a of the electrical power 312 to the at least one power storage device 220 (e.g., the at least one battery 232). The control circuitry 250 is configured to receive at least a second portion 312b of the electrical power 312 from the first circuitry 210 and to controllably provide at least some of the second portion 312b of the electrical power 312 to the second circuitry 220. The control circuitry 250 is in electrical communication with the at least one transistor 244 and is configured to provide transistor control signals 254 to the at least one transistor 244 and is in electrical communication with the at least one switch 246 and is configured to provide switch control signals 256 (e.g., binary on/off signals) to the at least one switch 246.

The control circuitry 250 of FIG. 3B comprises an input/output circuit 262, a microcontroller circuit 264, a feedback controller circuit 270, and at least one data storage device 280 (e.g., tangible storage, non-transitory storage, flash memory) in electrical communication with the feedback controller circuit 270 and configured to provide pre-programmed threshold values 282 to the feedback controller circuit 270. The input/output circuit 262 is in electrical communication with the microcontroller circuit 264 and is configured to controllably transmit the switch control signals 256 to the at least one switch 246 in response to control signals 268 received from the microcontroller circuit 264. In certain implementations, the microcontroller circuit 264 is configured to generate the control signals 268 and to controllably provide at least some of the second portion 312b of the electrical power 312 to the second circuitry 220.

As schematically illustrated in FIG. 3B, in certain implementations, the feedback controller circuit 270 is in electrical communication with the at least one transistor 244 and is configured to controllably transmit the transistor control signals 254 to the at least one transistor 244. The feedback controller circuit 270 is further configured to receive first sensor signals 272 (e.g., analog or digital battery voltage sense signals) indicative of the voltage V (e.g., sensed with respect to ground or Vₛₛ) of the at least one power storage device 230 (e.g., the voltage of the at least one battery 232), second sensor signals 274 (e.g., analog or digital supply voltage sense signals) indicative of the supply voltage V_{supply} (e.g., sensed with respect to ground or Vₛₛ) of the first circuitry 210, and third sensor signals 276 (e.g., analog or digital battery current sense signals) indicative of the electrical current flowing to the at least one power storage device 230 (e.g., through the at least one transistor 244 and the at least one switch 246). The feedback controller circuit 270 is further configured to provide (e.g., generate and transmit) the transistor control signals 254 to the at least one transistor 244 in response to the first sensor signals 272, the second sensor signals 274, and/or the third sensor signals 276. In certain implementations, the feedback controller circuit 270 is further configured to provide the transistor control signals 254 to the at least one transistor 244 in further response to a comparison of the first sensor signals, the second sensor signals, and/or the third sensor signals to corresponding pre-programmed threshold values (e.g., at least some of the pre-programmed threshold values 282 from the at least one data storage device 280). For example, the feedback controller circuit 270 is configured to actively regulate the first portion 312a of the electrical power 312 (e.g., a current during pre-charge) flowing to the at least one power storage device 230 and the second portion 312b of the electrical power 312 (e.g., a supply voltage V_{supply} of the first circuitry 210).

In certain implementations, the at least one transistor 244 is between the first circuitry 210 and the at least one power storage device 230 and is controllably connected and disconnected to the at least one power storage device 230 (e.g., by the at least one switch 246) such that the first portion 312a of the electrical power 312 (e.g., a pre-charge current) into the at least one power storage device 230 is controllably limited. Simultaneously with the first portion 312a of the electrical power 312 being provided to the at least one power storage device 230, at least some of the second portion 312b of the electrical power 312 is provided to the second circuitry 220 (e.g., a main supply voltage V_{supply} of the first circuitry 210 is significantly higher than the voltage of the at least one battery 232, provided that sufficient electrical power 312 is available from the first circuitry 210).

In certain implementations, if the at least one power storage device 230 is to be used to power the second circuitry 220, the microcontroller circuit 264 turns on (e.g., closes) the at least one switch 246 (e.g., by transmitting control signals 268 to the input/output circuit 262 which transmits the corresponding switch control signals 256 to the at least one switch 246). The feedback controller circuit 270 senses the voltage V of the at least one power storage device 230 (e.g., the battery voltage) by receiving the first sensor signals 272 (e.g., analog or digital battery voltage sense signals indicative of the voltage V of the at least one power storage device 230). If the voltage V is below a corresponding pre-programmed threshold value 282 (e.g., a first pre-programmed threshold value 282a above the minimum operating voltage Vₘᵢₙ of the at least one power storage device 230 and above the reset voltage Vᵣₑₛₑₜ of the control circuitry 250), the feedback controller circuit 270 enters a pre-charge mode of operation.

In the pre-charge mode of operation, the feedback controller circuit 270 of certain implementations controls the distribution of the electrical power 312 to the at least one power storage device 230 (e.g., the first portion 312a of the electrical power 312 flowing through the at least one transistor 244) and to the second circuitry 220 (e.g., the second portion 312b of the electrical power 312). As schematically illustrated by FIG. 3B, in the pre-charge mode of operation, the feedback controller circuit 270 receives the second sensor signals 274 (e.g., analog or digital V_{supply} sense signals) and the third sensor signals 276 (e.g., analog or digital battery current sense signals), and transmits the transistor control signals 254 in response to the second sensor signals 274 and the third sensor signals 276. In response to the transistor control signals 254, the at least one transistor 244 varies its impedance, such that the electrical power 312 is distributed between the first portion 312a and the second portion 312b. In certain implementations, the feedback controller circuit 270 controls an analog voltage of the first portion 312a received by the at least one transistor 244 such that the first portion 312a of the electrical power 312 received by the at least one power storage device 230 (e.g., electrical current flowing into the at least one battery 232 from the first circuitry 210) is limited to a pre-programmed maximum value (e.g., a second pre-programmed threshold value 282b) while the supply voltage V_{supply} (e.g., from the first circuitry 210) is not reduced below a pre-programmed maximum value (e.g., a third pre-programmed threshold value 282c). For example, this control can be achieved using negative feedback with two active control loops, one for each of the two conditions to be achieved (e.g., the first portion 312a of the electrical power 312 less than the second pre-programmed threshold value 282b and the supply voltage V_{supply} greater than the third pre-programmed threshold value 282c). During the pre-charge process, the voltage V rises.

If the voltage Vis above the corresponding pre-programmed threshold value 282 (e.g., above both the minimum operating voltage Vₘᵢₙ of the at least one power storage device 230 and above the reset voltage Vᵣₑₛₑₜ of the control circuitry 250), the at least one power storage device 230 is in a non-discharged state. In certain implementations, during time periods in which the first circuitry 210 is receiving electrical power 312, the third circuitry 240 is further configured to provide the received power 312 from the first circuitry 210 to the at least one power storage device 230 and to the second circuitry 220. During time periods in which the first circuitry 210 is not receiving electrical power 312, the third circuitry 240 (e.g., the feedback controller circuit 270) is further configured to keep the at least one transistor 244 on continually such that electrical power from the at least one power storage device 230 flows to the second circuitry 220 via the control circuitry 250. For example, after the voltage V rises above the corresponding pre-programmed threshold value 282 during the pre-charge operation, the feedback controller circuit 270 can exit the pre-charge mode of operation and keep the at least one transistor 244 turned on continually such that the second circuitry 220 operates using previously-stored electric power from the at least one power storage device 230.

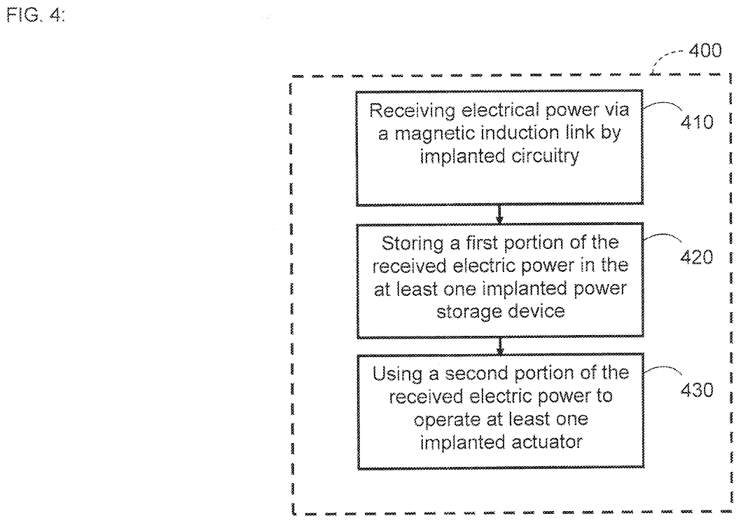

FIG. 4 is a flow diagram of an example method 400 in accordance with certain implementations described herein. In an operational block 410, the method 400 comprises receiving electric power via a magnetic induction link, the electric power received by circuitry implanted on or within a recipient's body from circuitry external to the recipient's body. For example, the magnetic induction link can transfer electric power from an external portion of a medical device or system (e.g., an auditory or visual prosthesis system; cardiac pacemaker or defibrillator system) with the electric power received by an internal portion (e.g., implanted component) of the medical device or system.

In an operational block 420, the method 400 further comprises, while at least one power storage device implanted on or within the recipient's body is discharged to a voltage below a minimum operating voltage of the at least one power storage device, storing a first portion of the received electric power in the at least one power storage device. For example, the at least one power storage device can be part of the internal portion of the medical device or system.

In an operational block 430, the method 400 further comprises, simultaneously with said storing the first portion, using a second portion of the received electric power to operate at least one actuator implanted on or within the recipient's body. For example, the at least one actuator can be part of the internal portion of the medical device or system (e.g., stimulation assembly of an auditory or visual prosthesis system; electrodes of a cardiac pacemaker or defibrillator system).

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having at least a portion of the received power available for use by the implanted device during time periods in which the at least one power storage device of the implanted device unable to provide electrical power for operation of the implantable medical device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
at least one housing configured to be implanted on or within a recipient's body;
first circuitry within the at least one housing, the first circuitry configured to wirelessly receive power from a device external to the recipient's body;
second circuitry within the at least one housing, the second circuitry configured to provide stimulation signals to a portion of the recipient's body;
at least one power storage device within the at least one housing, the at least one power storage device having a discharged state in which the at least one power storage device is discharged to a voltage below a minimum operating voltage of the at least one power storage device; and
third circuitry within the at least one housing, the third circuitry configured to, while the at least one power storage device is in the discharged state, controllably distribute the power simultaneously to both the second circuitry and the at least one power storage device.

2. The apparatus of claim 1, wherein the third circuitry is configured to actively regulate a first portion of the power distributed to the at least one power storage device and a second portion of the power distributed to the second circuitry.

3. The apparatus of claim 1, wherein the third circuitry comprises at least one resistor and at least one transistor in parallel electrical communication with the at least one resistor, the at least one transistor configured to switch between an open state and a closed state in response to transistor control signals, the at least one transistor in the closed state forming an electrical short across the at least one resistor.

4. The apparatus of claim 3, wherein the third circuitry is configured to generate the transistor control signals in response, at least in part, to the voltage of the at least one power storage device.

5. The apparatus of claim 1, wherein the third circuitry comprises at least one transistor and a feedback controller circuit, the at least one transistor configured to vary an impedance of the at least one transistor in response to transistor control signals from the feedback controller circuit.

6. The apparatus of claim 5, wherein the feedback controller circuit is configured to receive first sensor signals indicative of the voltage of the at least one power storage device, second sensor signals indicative of the supply voltage of the first circuitry, and third sensor signals indicative of the electrical current flowing to the at least one power storage device, the feedback controller circuit further configured to provide the transistor control signals to the at least one transistor in response, at least in part, to the first sensor signals, the second sensor signals, and/or the third sensor signals.

7. The apparatus of claim 6, wherein the feedback controller circuit is further configured to provide the transistor control signals to the at least one transistor in further response to a comparison of the first sensor signals, the second sensor signals, and/or the third sensor signals to corresponding pre-programmed threshold values.

8. The apparatus of claim 1, wherein the third circuitry is further configured to, while the at least one power storage device is in the discharged state, control an electric current flowing to the at least one power storage device such that an electric voltage applied to the second circuitry is above a threshold operating voltage of the second circuitry.

9. The apparatus of claim 1, wherein the third circuitry is configured to, while the at least one power storage device is in the discharged state, controllably distribute the electric power in response to the electric current flowing to the at least one power storage device, the electric voltage applied to the second circuitry, and/or the voltage of the at least one power storage device.

10. The apparatus of claim 2, wherein the third circuitry further comprises at least one switch configured to switch between an open state and a closed state in response to switch control signals, the at least one switch in the closed state providing the first portion of the electrical power to the at least one power storage device.

11. The apparatus of claim 1, wherein the at least one power storage device has at least one non-discharged state in which the at least one power storage device is not discharged to a voltage below the minimum operating voltage of the at least one power storage device, the third circuitry further configured to, while the at least one power storage device is in the non-discharged state, provide the power from the first circuitry to the at least one power storage device and to the second circuitry.

12. The apparatus of claim 1, wherein the second circuitry is configured to, while the at least one power storage device is in the discharged state, provide the stimulation signals to the portion of the recipient's body.

13. The apparatus of claim 1, wherein the at least one power storage device comprises at least one lithium-ion battery.

14. The apparatus of claim 1, wherein the apparatus comprises a totally implantable cochlear implant system.

15. A method comprising:
receiving electric power via a magnetic induction link, the electric power received by circuitry implanted on or within a recipient's body from circuitry external to the recipient's body;
while at least one power storage device implanted on or within the recipient's body is discharged to a voltage below a minimum operating voltage of the at least one power storage device, storing a first portion of the received electric power in the at least one power storage device; and
simultaneously with said storing the first portion, using a second portion of the received electric power to operate at least one actuator implanted on or within the recipient's body.

16. The method of claim 15, wherein the magnetic induction link is transcutaneous.

17. The method of claim 15, wherein the at least one power storage device comprises at least one battery.

18. The method of claim 15, wherein the circuitry implanted on or within the recipient's body, the at least one power storage device, and the at least one actuator are portions of an implantable medical system.

19. The method of claim 18, wherein the implantable medical system comprises an auditory prosthesis system, a visual prosthesis system, cardiac pacemaker system, or a cardiac defibrillator system.

20. The method of claim 15, further comprising operating the at least one actuator using previously-stored electric power from the at least one power storage device while the at least one power storage device is not discharged to a voltage below the minimum operating voltage.

21. The method of claim 15, further comprising actively regulating the first portion of the received electric power and the second portion of the received electric power.

22. The method of claim 21, wherein said actively regulating is in response, at least in part, to first sensor signals indicative of the voltage of the at least one power storage device, second sensor signals indicative of a supply voltage of the circuitry implanted on or within the recipient's body, and third sensor signals indicative of an electrical current flowing to the at least one power storage device.

23. The method of claim 15, wherein said actively regulating comprises using negative feedback with a first active control loop having the first portion of the received electrical power less than a corresponding threshold value and a second active control loop having a supply voltage provided to the at least one actuator greater than a corresponding threshold value.

24. An apparatus comprising:
a power transfer circuit configured to wirelessly receive power;
at least one battery configured to store at least a first portion of the power received by the power transfer circuit, the at least one battery having a discharged state and at least one non-discharged state;
at least one actuator configured to operate using power from the power transfer circuit and/or power from the at least one battery; and
a controller configured to, while the at least one battery is in the discharged state, controllably distribute the power received by the magnetic induction circuitry simultaneously to both the at least one battery and the at least one actuator.

25. The apparatus of claim 24, wherein the controller is further configured to, while the at least one battery is in the discharged state, control an electric current flowing to the at least one battery such that an electric voltage applied to the actuator is above a threshold operating voltage of the actuator.

26. The apparatus of claim 24, wherein the at least one battery has a minimum operating voltage below which the at least one battery is unable to output stored power, the at least one battery in the discharged state having a voltage below the minimum operating voltage and the battery in the at least one non-discharged state having a voltage above the minimum operating voltage.
